(19) United States
(12) Patent Application Publication
(10) Pub. No.: US 2014/0235494 A1
(43) Pub. Date: Aug. 21, 2014

(54) Methods, Kits and Computer Program Products using Hepatocellular Carcinoma (HCC) Biomarkers

(71) Applicant: The University of North Carolina at Chapel Hill, Chapel Hill, NC (US)

(72) Inventors: Monica Schmidt, Graham, NC (US); Michael Fried, Chapel Hill, NC (US)

(73) Assignee: The Universify of North Carolina at Chapel Hill, Chapel Hill, NC (US)

(21) Appl. No.: 14/346,360

(22) PCT Filed: Sep. 19, 2012
(86) PCT No.: PCT/US2012/055987

§ 371 Date: Mar. 21, 2014

Related U.S. Application Data
(60) Provisional application No. 61/537,265, filed on Sep. 21, 2011.

(51) Int. Cl. G01N 33/68

(52) U.S. Cl. CPC G01N 33/6893
USPC 506/9; 506/18

(57) ABSTRACT

Methods of determining the presence or risk of liver disease or other diseases in a subject include measuring a level of a saccharide selected from N-acetylneuraminic acid, N-acetylglucosamine, N-acetylgalactosamine, or a combination thereof on one or more glycosylated proteins and the total amount of alpha-fetoprotein in a biological sample from the subject. Glycosylated proteins include but are not limited to vascular endothelial growth factor, lymphatic vessel endothelial hyaluronan receptor, E-cadherin, alpha 1 acid glycoprotein, glypican-3, galectin-3, and any combination thereof. The presence or risk of disease in the subject is determined responsive to a value based on the level of glycosylation and amount of alpha-fetoprotein.

### STATEMENT OF PRIORITY

This application claims the benefit of U.S. Provisional Application Ser. No. 61/537,265, filed Sep. 21, 2011, the entire contents of which is incorporated by reference herein.

### STATEMENT OF FEDERAL SUPPORT

This invention was made, in part, with government support under grant numbers DK066144 and UL1TR000083 from the National Institutes of Health. The United States government has certain rights to this invention.

### FIELD OF THE INVENTION

The present invention relates to biomarkers associated with liver disease and other diseases, and more particularly, to methods and kits for using biomarkers to determine a presence or risk of disease in a subject.

### BACKGROUND

Hepatocellular carcinoma (HCC) is a frequent and unpredictable complication of liver disease, e.g., due to chronic hepatitis C and obesity-associated liver disease. Early diagnosis (Barcelona Clinic Liver stage 0 or A) is associated with better outcomes due to the availability of therapeutic modalities such as radiofrequency ablation, chemoembolization and liver transplantation in select cases. The annual incidence of HCC in subjects with cirrhosis has been estimated to be between 0.8% and 6% per year. HCC is one of the few cancers with an increasing incidence in the United States, largely due to the increase in cirrhosis from chronic Hepatitis C and obesity-associated liver diseases. Alpha-fetoprotein (AFP) measured in serum has been used to screen for HCC; however, its relatively low sensitivity (60%-80%) and specificity (70%-90%) has limited its usefulness, and current guidelines from the American Association for the Study of Liver Diseases eschews AFP in favor of cross-sectional imaging, such as ultrasound, CT scans and MRI scans. However, the sensitivity of ultrasound is only 60%-70%. The expense of these imaging tests, which are generally recommended at intervals of six months for patients with cirrhosis, further hampers adherence to the recommended screening.

### SUMMARY OF EMBODIMENTS OF THE INVENTION

The present invention is based on the discovery that the levels of certain saccharides on particular glycosylated proteins can serve as biomarkers for the presence and/or risk of liver disease of other diseases in a subject. Glycosylation levels of these biomarker proteins can also serve to predict the risk of disease as well as staging of disease. In particular, the inventors have shown that the levels of one or more of N-acetylneuraminic acid (sialic acid), N-acetylglucosamine, N-acetylgalactosamine, or any combination thereof on one or more of the identified biomarker proteins is indicative of the presence, stage, risk, and/or responsiveness of a disease.

Thus, one aspect of the present invention relates to a method for detecting the presence of a liver disease in a subject, comprising:

(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of alpha fetoprotein (AFP) in the biological sample; and
(c) determining that a liver disease is present based on the level of saccharide and amount of AFP.

In another aspect, the present invention relates to a method for detecting the presence of cancer in a subject, comprising:

(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of AFP in the biological sample; and
(c) determining that cancer is present based on the level of saccharide and amount of AFP.

In an additional aspect, the present invention relates to a method for staging a liver disease or disorder in a subject, comprising:

(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of AFP in the biological sample; and
(c) staging a liver disease or disorder in the subject based on the level of saccharide and amount of AFP. In some embodiments, the liver disease can be HCC, cirrhosis, and/or fibrosis.

In another aspect, the present invention relates to a method for determining the risk of developing a liver disease in a subject, comprising:

(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of AFP in the biological sample; and
(c) determining the risk of developing a liver disease based on the level of saccharide and amount of AFP.

In a further aspect, the present invention relates to a method for determining responsiveness to treatment in a subject having a liver disease, comprising:

(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of AFP in the biological sample; and
(c) determining responsiveness to treatment in the subject based on the level of saccharide and amount of AFP.

One aspect of the invention relates to an assay for detecting the presence of a liver disease in a subject, comprising:

a) measuring the level of a saccharide selected from N-acetylneuraminic acid, N-acetylglucosamine, N-acetylgalactosamine, or any combination thereof on one or more glycosylated proteins in a biological sample from a subject;
b) measuring the amount of AFP in the biological sample; and
b) determining that a liver disease is present in the subject based on the level of saccharide and amount of AFP.

One aspect of the invention relates to a histological method for staging a liver disease in a subject, comprising measuring the level of a saccharide selected from N-acetylneuraminic acid, N-acetylglucosamine, N-acetylgalactosamine, or any combination thereof in a tissue sample from a subject and staging the liver disease in the subject based on the level of saccharide. The disease stage correlates with the level of glycosylation of proteins in the tissue sample. Generally, the higher the level of saccharide, the higher the stage of the liver disease. One of skill in the art can readily use this correlation to stage liver diseases.

One aspect of the invention relates to kits for carrying out the disclosed methods. In some embodiments, the kits are immunoassay kits. In some embodiments, the immunoassay is a sandwich-based design. In some embodiments, the kit comprises: (a) one or more reagents for measuring the level of a saccharide selected from N-acetylneuraminic acid, N-acetylglucosamine, N-acetylgalactosamine, or any combination thereof on one or more glycosylated proteins in a biological sample from a subject; and (b) one or more reagents for measuring the amount of AFP in a biological sample from a subject. In one embodiment, the kit comprises one or more ligands that selectively bind to each of the one or more glycosylated proteins, e.g., one ligand specific for each glycosylated protein to be assayed.

In a further aspect, the present invention relates to a computer program product for carrying out the methods of the invention, the computer program product comprising a computer readable media having computer readable program code embodied therein, the computer readable program code comprising: computer readable program code configured to measure a level of a saccharide selected from N-acetylneuraminic acid, N-acetylglucosamine, N-acetylgalactosamine, or any combination thereof on one or more glycosylated proteins in a biological sample from the subject and the amount of AFP in the biological sample; and computer readable program code configured to determine a result based on the level of saccharide and amount of AFP.

Another aspect of the invention relates to a system for carrying out the methods of the invention, the system comprising:

a) a determination module configured to receive a biological sample from a subject and measure the level of a saccharide selected from N-acetylneuraminic acid, N-acetylglucosamine, N-acetylgalactosamine, or any combination thereof on one or more glycosylated proteins in the biological sample, and measure the amount of AFP in the biological sample;
b) a storage device configured to store data output from said determination module; and
c) a display module for displaying a content based in part on the data output from said determination module.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a schematic diagram of methods, systems and computer program products according to some embodiments of the present invention.

FIGS. 2-3 are flowcharts illustrating operations according to some embodiments of the present invention.

FIGS. 4A-4B are schematic diagrams of an assay according to some embodiments of the present invention.

FIG. 5 is a graph of the median fluorescent intensity for VEGF+biotinylated Dolichos biflorus agglutinin 1 lectin (glycan specificity: N-acetylgalactosamine) for a cirrhotic patient control group and a HCC patient group.

FIG. 6 is a graph of the median fluorescent intensity for LYVE-1+biotinylated Dolichos biflorus agglutinin 1 lectin (glycan specificity: N-acetylgalactosamine) for a cirrhotic patient control group and a HCC patient group.

FIG. 7 shows the performance of a selected model of the invention.

FIG. 8 shows histological analysis of liver tissue with wheat germ agglutinin.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention.

### DEFINITIONS

As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

It will be understood that, although the terms “first,” “second,” etc. may be used herein to describe various elements, and these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, embodiments of the present invention may take the form of a computer program product on a computer-usable or computer-readable non-transient storage medium having computer-usable or computer-readable program code embodied in the medium for use by or in connection with an instruction execution system.

“Altered level” or “altered levels” as used with respect to biomarker proteins herein refer to an increased level (e.g., a 10%, 20%, 30%, 40%, 50%, 100% increase, or more) or a decreased level (e.g., a 10%, 20%, 30%, 40%, 50%, 100% decrease, or more) in the quantity of one or more biomarker proteins and/or the level of glycosylation of one or more biomarker proteins detectable in or via a biological sample removed or derived from a subject, as compared to a level or levels of one or more biomarker proteins and/or level of glycosylation of one or more biomarker proteins in a control. A control sample includes a biological sample from a corresponding subject not afflicted with a liver disease such as HCC, a biological sample having some level of liver disease (e.g., cirrhosis) but not afflicted with HCC, or a biological sample from a non-diseased tissue or non-diseased portion of a tissue from the same subject. A presence or absence of a detectable amount of one or more biomarker proteins may also be considered an altered level.

“Biological sample” as used herein refers to any material taken from the body of a subject that may carry the target compound or compounds of the tests described herein, including both tissue samples and biological fluids such as blood samples, saliva samples, urine samples, etc.

“Blood sample” as used herein refers to whole blood or any fraction thereof that may contain detectable levels of biomarker proteins therein (if biomarker proteins are present in the whole blood sample from which said fraction is obtained), and in particular embodiments refers to a blood serum or blood plasma sample.

“Diagnosing,” “prognosing,” “risk,” “screening,” or “responsiveness” as used herein means providing an indication that a subject may be afflicted with or at risk of developing a disease, e.g., a liver disease such as HCC, and includes other terms such as screening for a disease, providing a risk assessment for disease, determining responsiveness to treatment, etc. It will be appreciated that no such technique is perfect and that such diagnosis, prognosis or the like may be confirmed by other procedures such as physical examination, imaging, histological examination of tissue samples, etc. The term “prognosing” or “responsiveness” as used herein includes providing an assessment or indication of disease in response to treatment (such as surgery, radiation therapy, chemotherapy, and combinations thereof) after initial diagnosis, as an indication of the efficacy of the treatment, risk of the disease returning, severity of disease following treatment, or the like.

“Marker protein,” “marker” or “biomarker” as used herein refers to any protein that can be detected, directly or indirectly (e.g., via an analog, metabolite, fragment or breakdown product including post-translational modifications such as glycosylation, fucosylation, methylation and/or acetylation) in a biological sample from a subject, an increase or decrease of the amount of which, compared to amounts found in similar subjects without disease, with a lower level of disease, or non-diseased tissue within the same subject (e.g., controls), is indicative of the presence or risk of liver disease such as HCC in a subject. Biomarker proteins of this invention include any protein listed in Table 1 herein. The analog, metabolite, fragment or breakdown product of the biomarker protein may or may not possess the functional activity of the biomarker protein listed.

“Panel test” or “multivariate assay” as described herein refers to a group of individual laboratory tests that are related in some way, including, but not limited to, the medical condition they are designed to detect (e.g., liver disease or HCC), the specimen type (e.g., blood), and the methodology employed by the test (e.g., detection of altered level of a target protein or proteins).

The term “simultaneous” or “simultaneously,” with respect to an assay, refers to two or more events that are sufficiently close in time to produce results at about the same time (e.g., within seconds or minutes of each other).

“Subjects” as described herein include human subjects and “patients” as well as other mammals in veterinarian or research settings, including mice. The subjects may be male or female and may be of any race or ethnicity, including but not limited to Caucasian, African-American, African, Asian, Hispanic, Indian, etc. The subjects may be of any age, including newborn, neonate, infant, child, adolescent, adult, and geriatric. Subjects may also include animal subjects, particularly mammalian subjects such as dog, cat, horse, mouse, rat, etc., screened for veterinary medicine or pharmaceutical drug development purposes. Subjects include but are not limited to those who may have, possess, have been exposed to, or have been previously diagnosed as afflicted with one or more risk factors for a liver disease or cancer. Risk factors include age, gender, race, smoking, diet, obesity, diabetes, work exposure, family history, alcohol and drug use and liver conditions such as hepatitis infection (A, B or C), autoimmune hepatitis, cryptogenic hepatitis, or other etiology that may result in liver cirrhosis. These risk factors may be considered in combination with the disclosed methods of detecting liver conditions such as HCC or cancer for a diagnosis, prognosis or screening. The disclosed methods of detecting liver conditions such as HCC or cancer for a diagnosis, prognosis or screening may also be used in combination with other diagnostic methods, including, but not limited to, scanning of the liver by an ultrasound or CT scan of the abdomen, detection of bilirubin and other substances, physical signs of jaundice, performing a biopsy, and screening for other biomarkers or other indicators of the possibility of disease (e.g., MELD (model for end-stage liver disease) score). Those skilled in the art will appreciate that this listing of other methods of detecting disease for a diagnosis, prognosis or screening is by no means exhaustive, and is but a small sampling of the other possible diagnostic methods that can easily be combined with the disclosed methods for purposes of diagnosis, prognosis or screening for HCC or other liver conditions.

A “value” or “level” may be an amount of biomarker or amount of glycosylation of a biomarker in a sample, a score based on the detected amounts of biomarkers in a sample, a percentile based on a population of patients, a quantitative amount or semi-quantitative amount of biomarker in a sample, a ratio of glycosylation to total biomarker, or other suitable quantities based on the detected biomarkers.

The “level of a saccharide” on a glycosylated protein refers to a quantitative or semi-quantitative amount of the stated saccharide in the glycan groups on a protein. The measured level is not limited to a saccharide in a specific position in the glycan groups or limited in its linkage to other specific saccharides. The “level” can be determined using any ligand that specifically binds to the saccharide of interest. A measure of the amount of ligand bound indicates the level of the saccharide. In some embodiments, the level of saccharide can be determined quantitatively by comparing the amount of ligand binding to a standard curve. In other embodiments, the level of saccharide can be determined semi-quantitatively using the amount of ligand binding without comparing the value to a standard curve (e.g., as measured in median fluorescent intensity using a fluorescently labeled ligand and compared to a negative control).

A “glycosylated protein” is any protein having one or more saccharide or glycan (polysaccharide) groups attached thereto.

The “amount of AFP” in a biological sample refers to the total amount of AFP protein in the sample. The amount is quantitated by comparison to a standard curve and is reported in ng/mL.

The term “sensitivity” as used herein refers to the proportion of actual positives which are correctly identified as such.

The term “specificity” as used herein refers to proportion of actual negatives that are correctly identified as such.

The term “AUROC” refers to Area Under Receiver Operating Characteristic and is a combination of sensitivity and specificity used to illustrate the performance of a binary classifier system. AUROC is a common summary statistic for the goodness of a predictor in a binary classification task. It is equal to the probability that a predictor will rank a randomly chosen positive instance higher than a randomly chosen negative one. AUROC is calculated from a plot of the true positive rate (sensitivity) versus the false positive rate (one minus the specificity).

The term “positive predictive value” or “PPV” refers to the proportion of positive test results that are true positives.

The term “negative predictive value” or “NPV” refers to the proportion of negative test results that are true negatives.

### Methods

The present invention is based on the discovery that the levels of certain saccharides on particular glycosylated proteins can serve as biomarkers for the presence and/or risk of liver disease of other diseases in a subject. Glycosylation levels of these biomarker proteins can also serve to predict the risk of disease as well as staging of disease. In particular, the inventors have shown that the levels of one or more of N-acetylneuraminic acid, N-acetylglucosamine, N-acetylgalactosamine, or any combination thereof on one or more of the identified biomarker proteins is indicative of the presence, stage, risk, and/or responsiveness of a disease. One advantage of the present invention is the ability of the disclosed methods to detect disease without invasive and expensive techniques. Another advantage is the ability of the disclosed methods to detect disease earlier (at a potentially more treatable stage) then current techniques. Additionally, the disclosed methods are less costly than imaging of any type.

Thus, one aspect of the present invention relates to a method for detecting the presence of a liver disease in a subject, comprising:

(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of alpha fetoprotein (AFP) in the biological sample; and
(c) determining that a liver disease is present based on the level of saccharide and amount of AFP.

The liver disease can be any known liver disease, including without limitation HCC, cirrhosis, fibrosis, organ transplant rejection, veno-occlusive disease, sinusoidal obstruction syndrome, hepatitis virus (e.g., A, B, C, D, E, or G), non-alcoholic fatty liver disease, alcoholic liver disease, alcohol- or drug-induced hepatitis, steatohepatitis, autoimmune hepatitis, haemochromatosis, cholangiocarcinoma, metastatic cancers, Wilson's disease, Crigler-Najjar syndrome, primary sclerosing cholangitis, primary biliary cirrhosis, Budd-Chiari syndrome, protoporphyria, Gilbert's syndrome, rotor syndrome, glycogen storage disease type 2, hemangioma, hyperbilirubinemia, biliary atresia, Byler disease, Dubin-Johnson syndrome, alpha-1 antitrypsin deficiency, Caroli disease, Alagille syndrome, and progressive familial intrahepatic cholestasis.

In some embodiments, the subject is one that has a liver disease, has had a liver disease, or is at risk for a liver disease. A subject at risk for a liver disease, e.g., HCC or cirrhosis, can be one that has a family history of the disease, is genetically predisposed to the disease, or has symptoms, habits, or other diseases known to lead to the disease. In one embodiment, the subject has liver fibrosis. In another embodiment, the subject has liver cirrhosis. In a further embodiment, the subject has dysplastic lesions and/or regenerative nodules in the liver. In another embodiment, the subject has hepatitis A, B, or C. In a further embodiment, the subject is an alcoholic or drug user.

In another aspect, the present invention relates to a method for detecting the presence of cancer in a subject, comprising:

(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of AFP in the biological sample; and
(c) determining that cancer is present based on the level of saccharide and amount of AFP.

The cancer can be any known cancer, including without limitation melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified.

In an additional aspect, the present invention relates to a method for staging a liver disease in a subject, comprising:

(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of AFP in the biological sample; and
(c) staging a liver disease in the subject based on the level of saccharide and amount of AFP. In some embodiments, the liver disease can be HCC, cirrhosis, and/or fibrosis.

The method can be used to identify the stage of disease in a subject at risk for liver disease, exhibited symptoms of liver disease, or known to have a liver disease. The method can provide an indication of the stage of disease that correlates with any of the known scoring systems. For example, the Kanel scoring system for fibrosis/cirrhosis stages the fibrosis on a scale of 0 to 5: Stage 0: normal; Stage 1: portal expansion with fibrosis (<⅓ tracts with wisps of bridging.); Stage 2: bridging fibrosis; Stage 3: marked bridging fibrosis or early cirrhosis (with thin septa fibrosis); Stage 4: definite cirrhosis with <50% of biopsy fibrosis; Stage 5: definite cirrhosis with >50% of biopsy fibrosis. Other common scoring systems for fibrosis include the Metavir and Knodell systems. For HCC, numerous scoring systems exist, including the TNM, Okuda, Barcelona, and CLIP systems. The present method provides the ability to stage a liver disease without having to perform an invasive liver biopsy.

In another aspect, the present invention relates to a method for determining the risk of developing a liver disease in a subject, comprising:

(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of AFP in the biological sample; and
(c) determining the risk of developing a liver disease based on the level of saccharide and amount of AFP.

The method can be used to analyze the current condition of a subject (e.g., level of fibrosis/cirrhosis) and provide a determination of the potential for the subject to develop a specific liver disease (e.g., cirrhosis and/or HCC). Such a determination can be used to develop a treatment plan for the subject, such as when to start treatment and which treatment option to select. Another use is to determine appropriate imaging intervals for monitoring subjects. For example, high risk subjects might be imaged every three months while low risk subjects might be imaged every 12 months.

In a further aspect, the present invention relates to a method for determining responsiveness to treatment in a subject having a liver disease, comprising:

(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of AFP in the biological sample; and
(c) determining responsiveness to treatment in the subject based on the level of saccharide and amount of AFP.

The method can be used to develop a treatment plan for the subject based on the likely effectiveness of the treatment. The method can also be used to monitor the effectiveness of a treatment after it has been started.

In the above embodiments of the invention, the level of a saccharide on one or more glycosylated proteins can be measured. In certain embodiments, the level of a saccharide on 2, 3, 4, 5, or more glycosylated proteins is measured. In some embodiments, the glycosylated proteins are one or more proteins now known or identified in the future that is bound by a ligand that recognizes a specific saccharide, e.g., a plant lectin, antibody, or carbohydrate-binding protein. In one embodiment, the glycosylated proteins are one or more proteins selected from the list in Table 1 in any combination.

TABLE 1

| | |
|---|---|
| Vascular endothelial growth factor | Ig gamma-2 chain C region |
| Lymphatic vessel endothelial | Uncharacterized protein |
| hyaluronan receptor | |
| E-cadherin | Ig heavy chain V-I region |
| Alpha 1 acid glycoprotein | Ig kappa chain V-III region |
| Glypican-3 | Methionyl-tRNA synthetase, |
| | cytoplasmic |
| Galectin-3 | Immunoglobulin lambda-like |
| | polypeptide 5 |
| Spindle assembly abnormal | Zinc finger protein 136 |
| protein 6 homolog | |
| Alpha-2-macroglobulin | Vitronectin |
| Serotransferrin | Wee1-like protein kinase 2 |
| Serum albumin | Hemoglobin subunit beta |
| Alpha-1-antitrypsin | Keratin, type II cytoskeletal 1 |
| Haptoglobin | Myoferlin |
| Ig alpha-1 chain C region | Ig lambda chain V-I region |
| Alpha-1-acid glycoprotein 1 | Alpha-1B-glycoprotein |
| Apolipoprotein A-I | Ubiquitin-conjugating enzyme E2, J2 |
| | (UBC6 homolog, yeast), isoform |
| | CRA_d |
| Hemopexin | Neuronal pentraxin-2 |
| Ig kappa chain C region | Contactin-2 |
| Ig heavy chain V-III region | Centrosomal protein KIAA1731 |
| Ig mu chain C region | Fanconi anemia group B protein |
| | OS = Homo sapiens GN = FANCB |
| | PE = 1 SV = 1 |
| Alpha-1-acid glycoprotein 2 | Oxidised low density lipoprotein |
| | (Lectin-like) receptor 1, isoform |
| | CRA_c |
| Ig lambda-2 chain C region | Isoform Beta of Neuronatin |
| Serpin peptidase inhibitor, clade A | Fibroblast growth factor receptor |
| (Alpha-1 antiproteinase, antitrypsin), | substrate 2 |
| member 3, isoform CRA_b | |
| Kininogen-1 | |

In one embodiment, the glycosylated proteins are one or more proteins, e.g., 2, 3, 4, or 5 proteins, selected from vascular endothelial growth factor (VEGF), lymphatic vessel endothelial hyaluronan receptor (LYVE-1), E-cadherin, alpha 1 acid glycoprotein, glypican-3, galectin-3, or any combination thereof. In another embodiment, the glycosylated proteins are one or more proteins selected from VEGF, LYVE-1, E-cadherin, glypican-3, galectin-3, or any combination thereof. In a further embodiment, the glycosylated proteins are one or more proteins selected from VEGF, LYVE-1, E-cadherin, or any combination thereof. In an additional embodiment, the glycosylated proteins are one or more proteins selected from VEGF, E-cadherin, or the combination thereof.

In each aspect of the invention, the biological sample can be any tissue or fluid that will contain the biomarker proteins to be measured. In one embodiment, the biological sample can be whole blood, serum, plasma, bile, urine, tears, saliva, mucus, secretions, exudates, or tissue (e.g., biopsy tissue).

The level of a saccharide selected from N-acetylneuraminic acid, N-acetylglucosamine, and/or N-acetylgalactosamine on the one or more glycosylated proteins can be measured by any method known in the art. In some embodiments, immunoassays can be used. Numerous protocols for immunoassays using either polyclonal or monoclonal antibodies with established specificity are well known in the art. In certain embodiments, the immunoassays can be, e.g., immunofluorescence, immunohistochemistry, or chemiluminescence assays. In some embodiments, separate assays can be used to measure each glycosylated protein. In other embodiments, multiplex assays can be used to measure two or more glycosylated proteins simultaneously. Other assay techniques that can be used include, without limitation, chromatography, microscopy, mass spectroscopy, gel electrophoresis, and microarray analysis.

In some embodiments, the assay uses one or more ligands that specifically bind to the one or more glycoproteins. In certain embodiments, the ligands can be antibodies or antibody fragments (e.g., monoclonal antibodies) that specifically bind to the one or more glycoproteins. Such antibodies and antibody fragments (e.g., Fab, Fab′, F(ab′)₂, and Fv fragments, chimeric antibodies, domain antibodies, diabodies, vaccibodies, linear antibodies, single-chain antibody molecules, humanized antibodies, and multispecific antibodies formed from antibody fragments) are well known in the art and can also readily be developed by one of skill in the art. Such antibodies and antibody fragments can be used to separate the one or more proteins from the biological sample, prior to, during, or after the level of glycosylation is measured.

Antibodies can be coupled to a solid support (e.g., beads, particles, membranes, plates, slides or wells formed from materials such as glass, latex, plastic (e.g., polystyrene, polyethylene, polypropylene), metal, rubber, or ceramic) in accordance with known techniques. Coupling to the solid support can be done by any means known in the art, such as conjugation with a coupling agent, adsorption, non-covalent interactions, covalent interactions, and electrostatic interactions. The antibodies can be directly coupled to a detectable group or detection can proceed via a secondary reagent that specifically binds to the antibody. Antibodies can be conjugated to detectable groups such as radiolabels (e.g., ³⁵S, ¹²⁵I, ¹³¹I) enzyme labels (e.g., horseradish peroxidase, alkaline phosphatase), and fluorescence labels (e.g., fluorescein, streptavidin-phycoerythrin) in accordance with known techniques. Antibodies can also be linked indirectly to detectable groups (e.g., biotin linked to the antibody and streptavidin linked to the detectable group). Determination of the formation of an antibody/antigen complex in the methods of this invention can be by detection of, for example, precipitation, agglutination, flocculation, radioactivity, color development or change, fluorescence, luminescence, etc., as is well-known in the art.

In one embodiment, the level of a saccharide selected from N-acetylneuraminic acid, N-acetylglucosamine, and/or N-acetylgalactosamine is measured using a ligand that specifically binds to one or more of N-acetylneuraminic acid, N-acetylglucosamine, and/or N-acetylgalactosamine. The ligand can be an antibody, plant lectin, carbohydrate-binding protein, protein with lectin-like domain, or aptamer. The plant lectin can be, without limitation, wheat germ agglutinin lectin (which binds N-acetylneuraminic acid and N-acetylglucosamine), succinylated wheat germ agglutinin (which binds N-acetylglucosamine), soybean agglutinin lectin (which binds N-acetylgalactosamine), Dolichos biflorus agglutinin lectin (which binds N-acetylgalactosamine), or any combination thereof. In certain embodiments, the plant lectin is wheat germ agglutinin lectin. The ligand can be directly coupled to a detectable group or detection can proceed via a secondary reagent that specifically binds to the ligand. The ligand can be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein, streptavidin-phycoerythrin) in accordance with known techniques. Ligands can also be linked indirectly to detectable groups (e.g., biotin linked to the ligand and streptavidin linked to the detectable group).

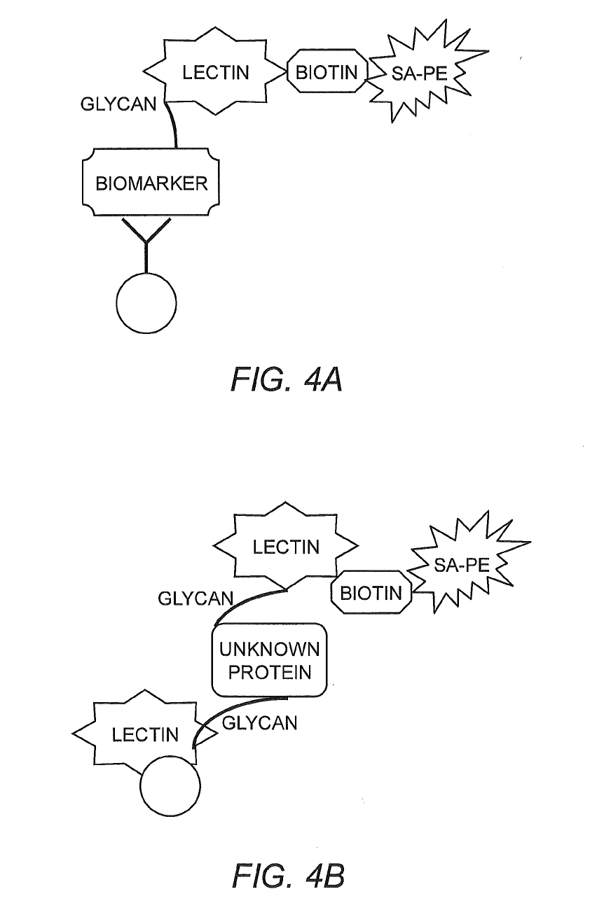

In some embodiments, the immunoassay is a sandwich-based design. Exemplary assays are illustrated in FIGS. 4A-4B. Antibodies (e.g., monoclonal antibodies) specific for each of the glycoproteins are conjugated to beads (e.g., magnetic beads or microspheres, e.g., Luminex® microspheres). Conjugation can be carried out using any technique known in the art to be suitable for antibodies (e.g., sulfo-NHS bio-conjugation). Each bead has its own unique detection signal (e.g., fluorescent signal) and a single bead set is chosen for each glycoprotein. Beads are then mixed together to form a master mix. In some embodiments, an antibody (e.g., a monoclonal antibody) specific for AFP is conjugated to a bead and included in the master mix. The biological sample (e.g., serum) can be diluted (e.g., in PBS) from 1:1 to about 1:100 (e.g., 1:5, 1:10, 1:20, or 1:50). The diluted sample is incubated with the master mix. In some embodiments, the antibodies are pre-incubated with a blocking solution (e.g., LowCross-Buffer®, Carbo-Free™ Blocking Solution) to block antibody glycans. Once the glycoproteins to be assayed are captured, all other components in the sample are washed away. The beads are then incubated with a ligand specific for N-acetylneuraminic acid, N-acetylglucosamine, and/or N-acetylgalactosamine. The ligand is detectably labeled (e.g., with biotin). The mix is then incubated with a detection molecule (e.g., streptavidin-phycoerythrin). A signal is obtained for each bead set and represents the semi-quantitative amount of the saccharide bound to the protein surface. In the example of biotin/streptavidin-phycoerythrin detection, the signal can be expressed as median fluorescent intensity.

In some embodiments, the assay uses Luminex® xMAP or Magpix technology (Luminex Corporation, Austin, Tex., USA) or a comparable device. The Luminex® xMAP or Magpix technology is a bead-based analyzer that combines flow cytometry and enzyme-linked immunoassay (EIA) techniques. The lasers in the Luminex® or xMAP or Magpix analyzer first detect the bead number (i.e., associated glycoprotein). The amount of tagged target protein captured on each bead is quantified by a CCD laser (e.g., measuring the median fluorescence intensity). Many readings may be made for each protein/bead set, further enhancing precision of the assay. In some embodiments, at least about 100 readings are made for each protein/bead set.

In some embodiments, the amount of AFP (e.g., total AFP in the sample) is detected using polyclonal antibodies and the total amount is assessed using standard immunoassay techniques. AFP detection can be carried out in a separate container from the glycoprotein assay or in the same container.

In some embodiments, the level of a saccharide selected from N-acetylneuraminic acid, N-acetylglucosamine, and/or N-acetylgalactosamine on one or more glycoproteins and the total amount of AFP is combined to determine the presence of a liver disease or cancer, staging of a liver disease, determining the risk of developing a liver disease, and/or determining responsiveness to treatment in a subject having a liver disease. In some embodiments, the determining step is carried out using an empirically-based regression algorithm using the combination of biomarkers. The determining step can further comprise assigning a weighted coefficient to the levels of a saccharide selected from N-acetylneuraminic acid, N-acetylglucosamine, and/or N-acetylgalactosamine and the total amount of AFP, e.g., based on actual clinical experience. In one embodiment, the algorithm can take the form:

Y=β₀+β₁X₁+β₂X₂+β₃X₃

wherein:

Y=outcome;

β₀=constant (log odds when all biomarkers are set to zero);

X1=biomarker 1;

X2=biomarker 2;

X3=biomarker 3; and

Beta coefficients are the weights each biomarker contributes to predicting the outcome.

Beta coefficients are between 0-1 and are generated by “training” the algorithm with many observations and taking the average of all the beta coefficients for each observation. The beta coefficients are essentially the odds of having a first outcome (e.g., having a disease) versus a second outcome (e.g., not having the disease) based on the biomarker value. The more patients used and the more diverse the population during training, the more accurate and externally valid the algorithm becomes. Boosting may be used to improve the performance of the algorithm. This method will weight individual predictions that are correct (predicted disease=true disease) more heavily than an incorrect prediction (predicted no disease=true disease).

In certain embodiments, the algorithm is set up such that the outcome is a binary result of 0 or 1 wherein 0 equals no disease and 1 equals disease. In other embodiments, the outcome is a number between 0 and 1 wherein numbers closer to 0 indicate a lower risk of disease or a lower stage of disease and numbers closer to 1 indicate a higher risk of disease or a higher stage of disease.

In some embodiments, the determining step comprises comparing the level of a saccharide selected from N-acetylneuraminic acid, N-acetylglucosamine, and/or N-acetylgalactosamine on one or more glycoproteins in the biological sample and the amount of AFP in the biological sample to the level of N-acetylneuraminic acid, N-acetylglucosamine, and/or N-acetylgalactosamine on the one or more proteins and the amount of AFP in a control sample. In certain embodiments, the control sample can be from a disease-free tissue (e.g., a disease-free portion of the liver) in the same subject or from a control subject. In certain embodiments, the control sample can be an average value calculated from samples from a population of subjects. The alteration in the level of a saccharide selected from N-acetylneuraminic acid, N-acetylglucosamine, and/or N-acetylgalactosamine on one or more glycoproteins can be either an increase or a decrease compared to control levels depending on the particular protein being measured. For example, an increase in glycosylation of VEGF, LYVE-1, glypican-3, or galectin-3 and a decrease in glycosylation of E-cadherin is indicative of the presence of disease or the risk of disease. The combination of an alteration in the level of saccharide and an increase in total AFP compared to control values is an additional indication of the presence of disease or the risk of disease.

In some embodiments, the assays are carried out on biomarkers individually or in panels with one another or other additional biomarkers such as described herein. Where used in a panel test (such as a multiplex Luminex® microsphere test), the levels or amounts of the various biomarkers are optionally but preferably tested from the same biological sample obtained from the subject (e.g., by detecting the quantities or amounts of various proteins in the same blood/serum sample obtained from a patient).

The methods of the present invention provide both high sensitivity and high specificity. In some embodiments, the methods provide a sensitivity of at least 80%, e.g., at least 85%, 90%, 95%, 96%, 97%, 98%, 99%, or 100%. In some embodiments, the methods provide a specificity of at least 80%, e.g., at least 85%, 90%, 95%, 96%, 97%, 98%, 99%, or 100%. In other embodiments, the methods provide a sensitivity and a specificity of at least 80%, e.g., at least 85%, 90%, 95%, 96%, 97%, 98%, 99%, or 100%.

In some embodiments, the methods of the invention comprise further steps based on the outcome of the determination. In one embodiment, when the determination indicates that the subject has a liver disease or cancer or indicates the stage of the disease, the methods further comprise the step of administering to the subject a treatment for the liver disease or cancer. Treatments can include, without limitation, surgery, radiation, chemotherapy, antiviral therapy, liver transplantation, immunostimulation, change in diet, avoidance of alcohol and/or drugs, and supportive care.

In other embodiments, when the determination indicates the risk of developing liver disease or the responsiveness of the liver disease to treatment, the methods further comprise the step of selecting an appropriate treatment or avoidance of treatment based on the risk or responsiveness. In another embodiment, the methods further comprise the step of administering to the subject an additional test to confirm the presence and/or stage of the liver disease or cancer. Additional tests can include, without limitation, biopsies, blood tests, imaging techniques (e.g., X-rays, MRI, CT scans), and liver function tests. In another embodiment, when the determination indicates that the subject does not have a liver disease or cancer or indicates an early stage of disease, the methods further comprise the step of choosing not to administer a treatment or choosing a less severe treatment.

One aspect of the invention relates to an assay for detecting the presence of a liver disease in a subject, comprising:

a) measuring the level of a saccharide selected from N-acetylneuraminic acid, N-acetylglucosamine, N-acetylgalactosamine, or any combination thereof on one or more glycosylated proteins in a biological sample from a subject;
b) measuring the amount of AFP in the biological sample; and
b) determining that a liver disease is present in the subject based on the level of saccharide and amount of AFP.

One aspect of the invention relates to a histological method for staging a liver disease in a subject, comprising measuring the level of a saccharide selected from N-acetylneuraminic acid, N-acetylglucosamine, N-acetylgalactosamine, or any combination thereof in a tissue sample from a subject and staging the liver disease in the subject based on the level of saccharide. In one embodiment, the level of saccharide is measured using a lectin such as wheat germ agglutinin. The disease stage correlates with the level of glycosylation of proteins in the tissue sample. Generally, the higher the level of saccharide, the higher the stage of the liver disease. One of skill in the art can readily use this correlation to stage liver diseases.

Tissue samples (e.g., biopsies) can be prepared for histological analysis by any methods known in the art and as described herein. The level of saccharide can be measured using a ligand that specifically binds to N-acetylneuraminic acid, N-acetylglucosamine, N-acetylgalactosamine, or any combination. The ligand can be an antibody or a plant ligand. The plant lectin can be, without limitation, wheat germ agglutinin lectin, soybean agglutinin lectin, Dolichos biflorus agglutinin lectin, or any combination thereof. In certain embodiments, the plant lectin is wheat germ agglutinin lectin. The ligand can be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein, streptavidin-phycoerythrin) in accordance with known techniques.

### Kits

One aspect of the invention relates to kits for carrying out the disclosed methods. In some embodiments, the kits are immunoassay kits. In some embodiments, the immunoassay is a sandwich-based design. In some embodiments, the kit comprises: (a) one or more reagents for measuring the level of a saccharide selected from N-acetylneuraminic acid, N-acetylglucosamine, N-acetylgalactosamine, or any combination thereof on one or more glycosylated proteins in a biological sample from a subject; and (b) one or more reagents for measuring the amount of AFP in a biological sample from a subject. In one embodiment, the kit comprises one or more ligands that selectively bind to each of the one or more glycosylated proteins, e.g., one ligand specific for each glycosylated protein to be assayed.

In certain embodiments, the ligands can be antibodies or antibody fragments (e.g., monoclonal antibodies) that specifically bind to the one or more glycoproteins. Such antibodies and antibody fragments (e.g., Fab, Fab′, F(ab)₂, and Fv fragments; domain antibodies, diabodies; vaccibodies, linear antibodies; single-chain antibody molecules; and multispecific antibodies formed from antibody fragments) are well known in the art and can also readily be developed by one of skill in the art.

The antibodies can be conjugated to a solid support (e.g., beads, plates, slides or wells formed from materials such as latex or polystyrene) in accordance with known techniques. The antibodies can likewise be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein, streptavidin-phycoerythrin) or conjugated to intermediates (e.g., biotin) that bind detectable groups in accordance with known techniques.

In some embodiments, antibodies (e.g., monoclonal antibodies) specific for each of the glycoproteins are conjugated to beads (e.g., magnetic beads or microspheres, e.g., Luminex® microspheres). Each bead can have its own unique detection signal (e.g., fluorescent signal) and a single bead set is chosen for each glycoprotein. Beads can be mixed together to form a master mix. In some embodiments, an antibody (e.g., a monoclonal antibody) specific for AFP is conjugated to a bead and included in the master mix.

The kits can contain reagents (e.g., ligands) to assay the level of a saccharide on 2, 3, 4, 5, or more glycosylated proteins. In one embodiment, the glycosylated proteins are one or more proteins selected from the list in Table 1 in any combination. In one embodiment, the glycosylated proteins are one or more proteins, e.g., 2, 3, 4, or 5 proteins, selected from VEGF, LYVE-1, E-cadherin, alpha 1 acid glycoprotein, glypican-3, galectin-3, or any combination thereof. In another embodiment, the glycosylated proteins are one or more proteins selected from VEGF, LYVE-1, E-cadherin, glypican-3, galectin-3, or any combination thereof. In a further embodiment, the glycosylated proteins are one or more proteins selected from VEGF, LYVE-1, E-cadherin, or any combination thereof. In an additional embodiment, the glycosylated proteins are one or more proteins selected from VEGF, E-cadherin, or the combination thereof.

In some embodiments, the kits further comprise a ligand that specifically binds to one or more of N-acetylneuraminic acid, N-acetylglucosamine, and/or N-acetylgalactosamine. The ligand can be an antibody or a plant ligand. The plant lectin can be, without limitation, wheat germ agglutinin lectin, soybean agglutinin lectin, Dolichos biflorus agglutinin lectin, or any combination thereof. In certain embodiments, the plant lectin is wheat germ agglutinin lectin. The ligand can be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein, streptavidin-phycoerythrin) or one half of a binding pair that can be linked through the other half of the binding pair to detectable groups (e.g., biotin, streptavidin) in accordance with known techniques.

In some embodiments, the kits further comprise a ligand (e.g., polyclonal antibodies) specific for AFP.

The kits can further comprise other reagents for carrying out the disclosed methods. Other reagents can include, without limitation, blocking solutions (e.g., LowCross-Buffer®, Carbo-Free™ Blocking Solution), wash solutions, buffer solutions, detection molecules (e.g., streptavidin-phycoerythrin), controls, and standards (e.g., AFP).

Optionally, the kits can include components for carrying out assays with the additional use of detection devices for immunoassay, chemiluminescence, chromatography, spectrometry, electrophoresis, sedimentation, isoelectric focusing, or any combination thereof. Examples include, without limitation, filter plates and multi-well plates. Analysis may be carried out on a single sample or multiple samples.

In addition, the kit may optionally include instructions for performing the method or assay. Additionally the kit may optionally include depictions or photographs that represent the appearance of positive and negative results. In some embodiments, the components of the kit may be packaged together in a common container.

The kits can include material for carrying out assays on biomarkers individually or in panels with one another or other additional biomarkers such as described herein.

### Computer Programs and Systems

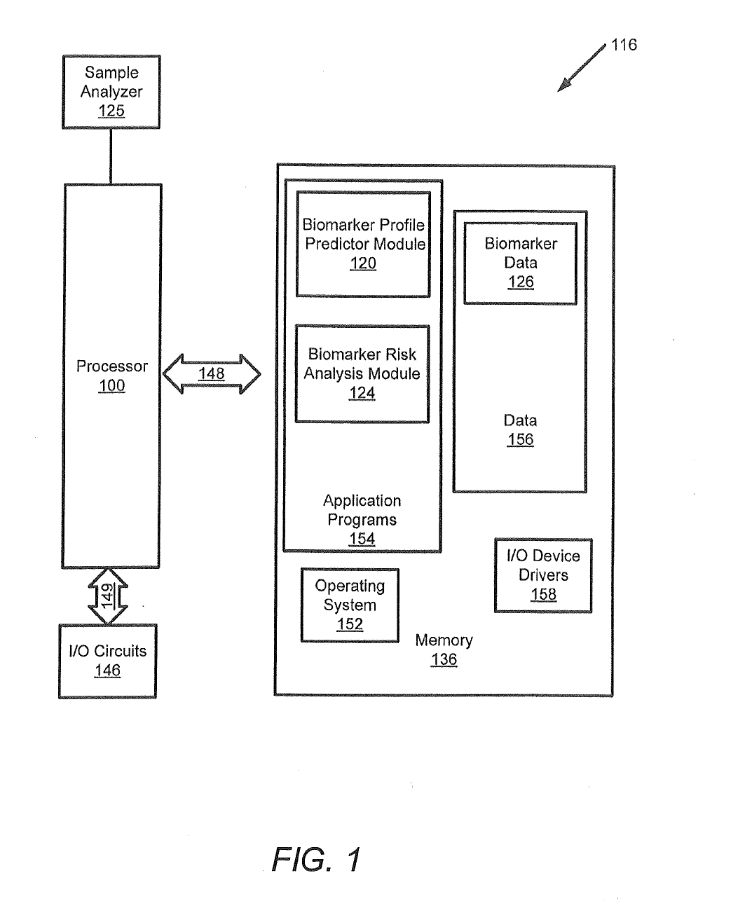

FIG. 1 illustrates an exemplary data processing system that can be included in devices operating in accordance with some embodiments of the present invention. As illustrated in FIG. 1, a data processing system 116, which can be used to carry out or direct operations includes a processor 100, a memory 136 and input/output circuits 146. The data processing system can be incorporated in a portable communication device and/or other components of a network, such as a server. The processor 100 communicates with the memory 136 via an address/data bus 148 and communicates with the input/output circuits 146 via an address/data bus 149. The input/output circuits 146 can be used to transfer information between the memory (memory and/or storage media) 136 and another component, such as a sample analyzer 125 (e.g., a Luminex® analyzer) for analyzing a sample. These components can be conventional components such as those used in many conventional data processing systems, which can be configured to operate as described herein.

In particular, the processor 100 can be a commercially available or custom microprocessor, microcontroller, digital signal processor or the like. The memory 136 can include any memory devices and/or storage media containing the software and data used to implement the functionality circuits or modules used in accordance with embodiments of the present invention. The memory 136 can include, but is not limited to, the following types of devices: cache, ROM, PROM, EPROM, EEPROM, flash memory, SRAM, DRAM and magnetic disk. In some embodiments of the present invention, the memory 136 can be a content addressable memory (CAM).

As further illustrated in FIG. 1, the memory (and/or storage media) 136 can include several categories of software and data used in the data processing system: an operating system 152; application programs 154; input/output device circuits 146; and data 156. As will be appreciated by those of skill in the art, the operating system 152 can be any operating system suitable for use with a data processing system, such as IBM®, OS/2®, AIX® or zOS® operating systems or Microsoft® Windows®2003, Windows2007 or WindowsXP operating systems Unix or Linux™. IBM, OS/2, AIX and zOS are trademarks of International Business Machines Corporation in the United States, other countries, or both while Linux is a trademark of Linus Torvalds in the United States, other countries, or both. Microsoft and Windows are trademarks of Microsoft Corporation in the United States, other countries, or both. The input/output device circuits 146 typically include software routines accessed through the operating system 152 by the application program 154 to communicate with various devices. The application programs 154 are illustrative of the programs that implement the various features of the circuits and modules according to some embodiments of the present invention. Finally, the data 156 represents the static and dynamic data used by the application programs 154, the operating system 152 the input/output device circuits 146 and other software programs that can reside in the memory 136.

The data processing system 116 can include several modules, including a biomarker profile predictor module 120, a biomarker risk analysis module 124, and the like. The modules can be configured as a single module or additional modules otherwise configured to implement the operations described herein for analyzing the biomarker profile of a sample. The data 156 can include biomarker profile data 126, for example, that can be used by the biomarker profile predictor module 120 and/or biomarker risk analysis module 124 to detect and/or analyze a biological sample and/or to control the sample analyzer 125.

While the present invention is illustrated with reference to the biomarker profile predictor module 120, the biomarker risk analysis module 124 and the biomarker data 126 in FIG. 1, as will be appreciated by those of skill in the art, other configurations fall within the scope of the present invention. For example, rather than being an application program 154, these circuits and modules can also be incorporated into the operating system 152 or other such logical division of the data processing system. Furthermore, while the biomarker profile predictor module 120 and the biomarker risk analysis module 124 in FIG. 1 is illustrated in a single data processing system, as will be appreciated by those of skill in the art, such functionality can be distributed across one or more data processing systems. Thus, the present invention should not be construed as limited to the configurations illustrated in FIG. 1, but can be provided by other arrangements and/or divisions of functions between data processing systems. For example, although FIG. 1 is illustrated as having various circuits and modules, one or more of these circuits or modules can be combined, or separated further, without departing from the scope of the present invention.

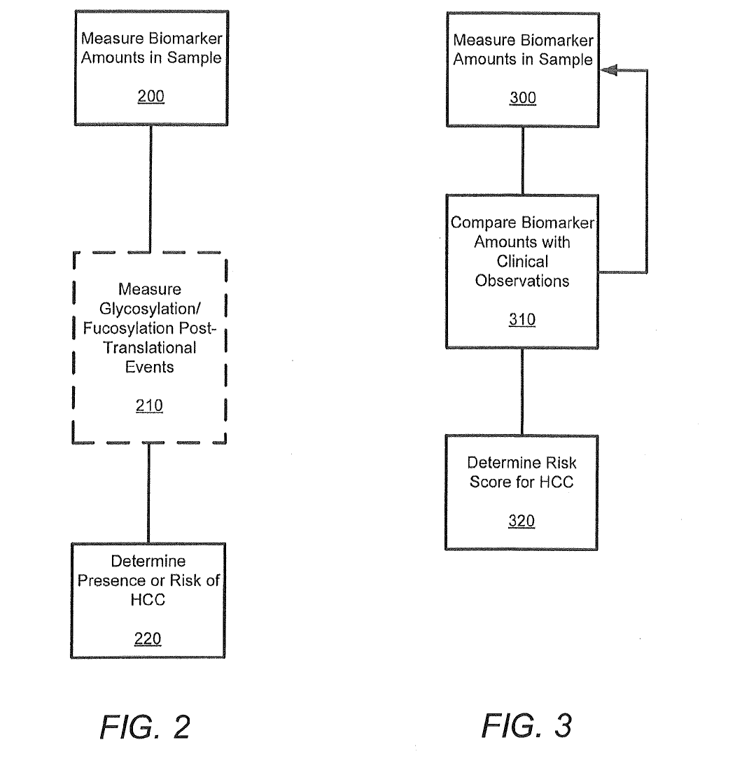

Accordingly, operations that may be performed by the biomarker profile predictor module 120 are illustrated in FIG. 2. An amount of some or all of the biomarkers in a biological sample, such as serum, is measured (Block 200) to determine a biomarker profile for a subject as described herein. The glycosylation/fucosylation post-translational events may optionally be measured (Block 210). The presence or risk of HCC may be determined based on the biomarker profile of the subject and optionally the glycosylation/fucosylation post-translational events (Block 220).

Operations that may be performed by the biomarker risk analysis module 123 are illustrated in FIG. 3. Biomarker amounts for some or all of the biomarkers of Table 1 may be measured (Block 300). The biomarker amounts may be compared with actual clinical observations (Block 310). Blocks 300 and 310 may be repeated for a sample group of patients to determine a “cut-off” or risk correlation between the biomarker amounts and the clinical observations (Block 320), e.g., using a multivariate analysis, which may include a support vector machine or logistic regression. A support vector machine and/or logistic regression may be partnered with machine learning methods. The risk correlation may be used to determine a presence or risk of HCC or other liver disease, e.g., as illustrated in FIG. 2, for a subject of unknown HCC or liver disease status. In some embodiments, the analysis may be combined with additional factors, such as age, gender, race, smoking, diet, obesity, diabetes, work exposure, family history, and liver conditions such as hepatitis A, B or C.

One aspect of the invention relates to a computer program product for detecting the presence of liver disease in a subject, the computer program product comprising a computer readable media having computer readable program code embodied therein, the computer readable program code comprising:

computer readable program code configured to measure a level of a saccharide selected from N-acetylneuraminic acid, N-acetylglucosamine, N-acetylgalactosamine, or any combination thereof on one or more glycosylated proteins in a biological sample from the subject and the amount of AFP in the biological sample; and
computer readable program code configured to determine that a liver disease is present based on the level of saccharide and amount of AFP.

In another aspect, the present invention relates to a computer program product for determining the presence of cancer in a subject, the computer program product comprising a computer readable media having computer readable program code embodied therein, the computer readable program code comprising:

computer readable program code configured to measure a level of a saccharide selected from N-acetylneuraminic acid, N-acetylglucosamine, N-acetylgalactosamine, or any combination thereof on one or more glycosylated proteins in a biological sample from the subject and the amount of AFP in the biological sample; and
computer readable program code configured to determine that cancer is present based on the level of saccharide and amount of AFP.

In an additional aspect, the present invention relates to a computer program product for staging a liver disease in a subject, the computer program product comprising a computer readable media having computer readable program code embodied therein, the computer readable program code comprising:

computer readable program code configured to measure a level of a saccharide selected from N-acetylneuraminic acid, N-acetylglucosamine, N-acetylgalactosamine, or any combination thereof on one or more glycosylated proteins in a biological sample from the subject and the amount of AFP in the biological sample; and
computer readable program code configured to stage a liver disease based on the level of saccharide and amount of AFP.

In another aspect, the present invention relates to a computer program product for determining the risk of developing a liver disease in a subject, the computer program product comprising a computer readable media having computer readable program code embodied therein, the computer readable program code comprising:

computer readable program code configured to measure a level of a saccharide selected from N-acetylneuraminic acid, N-acetylglucosamine, N-acetylgalactosamine, or any combination thereof on one or more glycosylated proteins in a biological sample from the subject and the amount of AFP in the biological sample; and
computer readable program code configured to determine the risk of developing a liver disease based on the level of saccharide and amount of AFP.

In a further aspect, the present invention relates to a computer program product for determining responsiveness to treatment in a subject having a liver disease, the computer program product comprising a computer readable media having computer readable program code embodied therein, the computer readable program code comprising: computer readable program code configured to measure a level of a saccharide selected from N-acetylneuraminic acid, N-acetylglucosamine, N-acetylgalactosamine, or any combination thereof on one or more glycosylated proteins in a biological sample from the subject and the amount of AFP in the biological sample; and computer readable program code configured to determine responsiveness to treatment in a subject having a liver disease based on the level of saccharide and amount of AFP.

Another aspect of the invention relates to a system for detecting the presence of a liver disease in a subject, the system comprising:

a) a determination module configured to receive a biological sample from a subject and measure the level of a saccharide selected from N-acetylneuraminic acid, N-acetylglucosamine, N-acetylgalactosamine, or any combination thereof on one or more glycosylated proteins in the biological sample, and measure the amount of AFP in the biological sample;
b) a storage device configured to store data output from said determination module; and
c) a display module for displaying a content based in part on the data output from said determination module, wherein the content comprises a signal indicative of the presence or absence of a liver disease.

Another aspect of the invention relates to a system for detecting the presence of cancer in a subject, the system comprising:

a) a determination module configured to receive a biological sample from a subject and measure the level of a saccharide selected from N-acetylneuraminic acid, N-acetylglucosamine, N-acetylgalactosamine, or any combination thereof on one or more glycosylated proteins in the biological sample, and measure the amount of AFP in the biological sample;
b) a storage device configured to store data output from said determination module; and
c) a display module for displaying a content based in part on the data output from said determination module, wherein the content comprises a signal indicative of the presence or absence of cancer.

In an additional aspect, the present invention relates to a system for staging a liver disease in a subject, the system comprising:

a) a determination module configured to receive a biological sample from a subject and measure the level of a saccharide selected from N-acetylneuraminic acid, N-acetylglucosamine, N-acetylgalactosamine, or any combination thereof on one or more glycosylated proteins in the biological sample, and measure the amount of AFP in the biological sample;
b) a storage device configured to store data output from said determination module; and
c) a display module for displaying a content based in part on the data output from said determination module, wherein the content comprises a signal indicative of the stage of the liver disease.

In another aspect, the present invention relates to a system for determining the risk of developing a liver disease in a subject, the system comprising:

a) a determination module configured to receive a biological sample from a subject and measure the level of a saccharide selected from N-acetylneuraminic acid, N-acetylglucosamine, N-acetylgalactosamine, or any combination thereof on one or more glycosylated proteins in the biological sample, and measure the amount of AFP in the biological sample;
b) a storage device configured to store data output from said determination module; and
c) a display module for displaying a content based in part on the data output from said determination module, wherein the content comprises a signal indicative of the risk of developing a liver disease.

In a further aspect, the present invention relates to a system for determining responsiveness to treatment in a subject having a liver disease, the system comprising:

a) a determination module configured to receive a biological sample from a subject and measure the level of a saccharide selected from N-acetylneuraminic acid, N-acetylglucosamine, N-acetylgalactosamine, or any combination thereof on one or more glycosylated proteins in the biological sample, and measure the amount of AFP in the biological sample;
b) a storage device configured to store data output from said determination module; and
c) a display module for displaying a content based in part on the data output from said determination module, wherein the content comprises a signal indicative of the responsiveness to treatment in the subject.

Embodiments according to the present invention are described in a non-limiting exemplary examples below.

### Example 1

### Bead Activation and Coupling

Luminex® beads are washed to remove antimicrobials and storage solution according to manufacturer procedures. The surface carboxyl groups are then activated via EDC and Sulfo-NHS to yield a long-lived intermediate Sulfo-NHS Ester. All unreacted EDC and Sulfo-NHS is then removed by several washes to reduce or prevent activation of carboxyl groups on the protein molecule which would result in protein-protein coupling rather than protein bead coupling. The surface carboxyl groups are then further activated via EDC and Sulfo-NHS to yield a long-lived intermediate Sulfo-NHS Ester.

The Luminex® beads are coupled with a capture antibody as follows. The activated Luminex® beads with Sulfo-NHS esters on the surface are combined with a protein solution and allowed to mix for two hours. Free amines on the protein side chains interact with the intermediate to form a covalent bond with the bead. An amount (e.g., 10 μg) of capture antibody is added to the re-suspended Luminex® as indicated in Table 2, The Luminex® beads are then washed in a wash buffer.

TABLE 2
Bead Coupling

| Bead | | | Volume | |
|---|---|---|---|---|
| Set | Capture Ab | Concentration | to Add | Manufacturer |
| 039 | Galectin-3 | 500 μg/mL | 20 μL | R&D Systems |
| 045 | E-Cadherin | 500 μg/mL | 20 μL | R&D Systems |
| 063 | LYVE-1 | 500 μg/mL | 20 μL | R&D Systems |
| 035 | Glypican-3 | 500 μg/mL | 20 μL | R&D Systems |
| 012 | VEGF | 500 μg/mL | 20 μL | R&D Systems |

Coupled Luminex® beads may be stored long term in accordance with the requirements of the respective coupled reagent. The Luminex® beads are counted on a hemocytometer and the antibody coupling is confirmed according to the manufacturer's instructions, e.g., by diluting the coupled bead stocks to a final concentration of 100 beads of each set per μL in PBS. An exemplary bead mixture and hemocytometer readings are shown in Table 3. A stock R-PE conjugated Goat Anti-Mouse IgG is reconstituted with 1 mL DI water and centrifuged if not it is clear. The stock is diluted as indicated in Table 4.

TABLE 3

| | Hemocytometer | Beads | PBS-1% BSA |
|---|---|---|---|
| Capture Ab | Count 10⁶ | μL | μL |
| LYVE-1 | 5.0 | 20 | 940 |
| E-CADHERIN | 5.0 | 20 | 940 |
| Galectin-3 | 5.0 | 20 | 940 |
| VEGF | 5.0 | 20 | 940 |
| E-Cadherin | 5.0 | 20 | 940 |

100% recovery of 5.0 x 10⁶ beads, add 20 μL of each bead set to 940 μL PBS-1% BSA
Calculation:
* # beads/1000 μL = bead/μL
**Bead/μL/100 = dilution factor
***1000/dilution factor = μL of initial coupled bead mix

TABLE 4
Stock PE-Anti-mouse IgG Antibody = .5 mg/mL

| | | | | Final |
|---|---|---|---|---|
| | PBS-1% | Volume | | Concentration |
| Tube # | BSA | (μL) | From Tube # | (μg/mL) |
| 1 | 492 | 8 | stock | 4 |
| 2 | 100 | 100 | 1 | 2 |
| 3 | 100 | 100 | 2 | 1 |
| 4 | 100 | 100 | 3 | 0.5 |
| 5 | 100 | 100 | 4 | 0.25 |
| 6 | 100 | 100 | 5 | 0.125 |
| 7 | 100 | 100 | 6 | 0.0625 |
| 8 | 200 | 0 | PBS stock | 0 (blank) |

Key:

| | | Anti-species |
|---|---|---|
| Bead Set | Capture Ab | PE Ab |
| 035 | Glypican-3 | Mouse |
| 039 | Galectin-3 | Mouse |
| 012 | VEGF | Mouse |
| 045 | E-Cadherin | Mouse |
| 063 | LYVE-1 | Mouse |

### Example 2

### HCC 5-Plex Glycomic Assay for Washed Capture Sandwich Immunoassay Using Magnetic Microspheres

Appropriate antibody-coupled microsphere sets are selected, and patient samples are diluted 1:5 with a Vector Labs Carbo-free blocker. The microspheres are washed and suspended by vortex and sonication for 20 seconds. A microsphere mixture (e.g., about 21 mL total) is prepared by diluting the coupled microsphere stocks to a final concentration of 100 microspheres of each set/μL in PBS (i.e., eliminate BSA). 50 μL of microsphere mixture is used for each reaction and is applied to the appropriate wells of a filter plate, and 500 μL of PBS is added to each background well and 50 μL of a sample is added to the appropriate wells. See Table 5.

TABLE 5

| | Stock Bead | μL of coupled | PBS Total |
|---|---|---|---|
| Protein | Concentration | beads to add | Volume |
| Glypican-3 | 5M | 420 | 18900 μL |
| LYVE-1 | 5M | 420 | (18.9 mL) |
| E-Cadherin | 5M | 420 | |
| VEGF | 5M | 420 | |
| Galectin-3 | 5M | 420 | |

individual single-plex mix: 20 μL in 980 μL PBS

The reactions are mixed gently and incubated for about one hour at room temperature on a plate shaker at 400 rpm in a dark environment. The wells are washed with 100 μL of PBS+Tween and vacuumed. The microspheres are resuspended in 50 μL of PBS only. The biotinylated lectins are reconstituted to 2 μg/mL by adding 500 μL ddH₂O, and working detection lectin is added to each well. See Table 6.

TABLE 6
Detection Lectin stock Reconstitution

| Detection Ab | ddH₂O | Final | WORKING |
|---|---|---|---|
| (biotinylated) | to add | Concentration | Lectin |
| ConA | 500 μL | 2 μg/μL | 5 μL in 995 μL ddH₂O |
| SBA | 500 μL | 2 μg/μL | separately for each |
| WGA | No Reconstitution | 2 μg/μL | lectin (total of 7 |
| | Necessary | | different working |
| DBA | 500 μL | 2 μg/μL | lectins to test |
| UEA | 500 μL | 2 μg/μL | individually against 5- |
| RCA | No Reconstitution | 2 μg/μL | plex mix) |
| | Necessary | | *Large volume: 50 μL |
| PNA | 500 μL | 2 μg/μL | lectin in 9950 μL |
| | | | ddH₂O |

enough for two full plates (Lectins purchased from Vector Laboratories (Burlingame, Calif. (U.S.A.))).

The reactions are mixed gently using a multi-channel pipettor, and the plate is incubated for one hour at room temperature on a plate shaker set to approximately 400 rpm in the dark. After washing the plate, the microspheres are resuspended in 50 μL of PBS only by gently pipetting up and down using the multi-channel pipettor. About 14 mL of 4 μg/mL streptavidin-R-phycoerythrin reporter is prepared by adding 32 μL of the 2.12 mg/mL stock streptavidin-R-phycoerythrin (Phycolink SA-PE product PJRS34) to 13968 μL of PBS only. About 50 μL of the diluted streptavidin-R-phycoerythrin is added to each well. The plate is covered and incubated for about 30 minutes at room temperature on a plate shaker set to approximately 400 rpm in the dark. The plate is washed and the microspheres are resuspended in 100 μL of PBS only by pipetting up and down using the multi-channel pipettor. The 100 μL sample mixture is transferred to a flat bottom round 96-well plate, and the sample mixtures are analyzed using a Luminex® analyzer according to the manufacturer's instructions.

### Example 3

Detection antibodies were coupled to magnetic Luminex® microspheres using a Sulfo-NHS bioconjugation method. The biomarkers of interest in a 50 μL serum sample were detected with the detection antibody coupled to the magnetic Luminex® microsphere in a diluted serum sample. E-Cadherin, VEGF, Glypican-3, Galectin-3 and LYVE-1 were the selected biomarkers. Biotinylated antibodies were added to the sample to detect an amount of biomarker in the serum sample.

Streptavidin-PE is added to the serum sample to detect an amount of each of the biomarkers to bind the biotinylated lectins. The final results are measured using the Luminex® Magpix instrument. The fluorescent signal of the magnetic Luminex® bead is programmed by number to indicate the protein biomarker of interest from the serum sample. The detection signal (biotinylated lectins+streptavidin-PE) is measured in median fluorescent intensity (MFI), indicating the level of glycan-lectin binding that takes place on the surface of each protein bound to the bead antibody complex.

The glycosylation and/or fucosylation post-translational events are evaluated for the chosen biomarkers (e.g., VEGF, E-Cadherin, Galectin-3, LYVE-1, Glypican-3 and combinations thereof). However, other markers may be used. These protein biomarkers are detected in serum using a multiplex that includes all four magnetic bead sets coupled to specific detection antibodies and combined into a master mix. An amount (e.g., about 50 μL) of this master mix is used per sample to probe the serum sample and detect the protein(s) of interest. Each biotinylated lectin may be applied to the sample individually and, in some embodiments, is not combined with other biotinylated lectins during the final detection process.

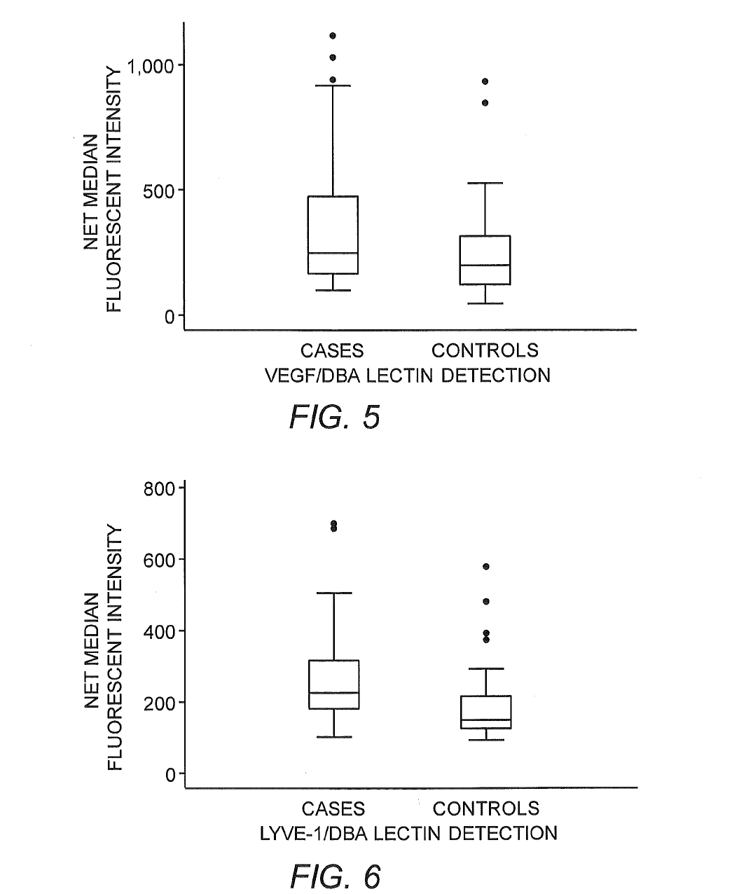

Post-translational modifications could be glycosylation/fucosylation of the protein or the loss thereof. The exact glycan changes may or may not be of interest; however, the median fluorescent intensity differences between cases and controls as detected with the biotinylated lectin and streptavidin-PE are analyzed. The differences between cases and controls are shown in FIGS. 5 and 6 and in Table 7 below based on measurements of quantitative differences in levels of glycosylation reported in MFI (median fluorescent intensity). The predictive value of the level of LYVE-1 N-acetylgalactosamine may be used to evaluate a risk score according to embodiments of the present invention such that increased levels of LYVE-1 N-acetylgalactosamine are associate with increased risk of HCC. The predictive value of the level of VEGF-1 N-acetylgalactosamine may be used to evaluate a risk score according to embodiments of the present invention such that increased levels of VEGF-1 N-acetylgalactosamine are associated with increased risk of HCC.

FIG. 5 is a graph of the differences between the mean values of VEGF N-acetylgalactosamine in cases of HCC versus cirrhotic controls, which was found to be significant at p=0.022 (p<0.05). FIG. 6 is a graph of the differences between the mean values of LYVE-1 N-acetylgalactosamine in cases of HCC versus cirrhotic controls, which was found to be significant at p=0.001 (p<0.05). Either logistic regression or support vector machine learning may be used to obtain an formula to classify patients. The data represented in the graph above represents a cohort of n=121 without use of an algorithm to optimize classification of cancer. Patients that are misclassified in the box plot shown in FIGS. 5 and 6 may be correctly classified by a logistic regression or support vector machine techniques.

TABLE 7
Raw Data Analysis (Non-Support Vector Machine Learning)

| | | | | LYVE-1- |
|---|---|---|---|---|
| | VEGF-CASES | VEGF-Controls | LYVE-1-CASES | Controls |
| Mean | 293.24 | 186.54 | 243.79 | 160.97 |
| SD | 276.14 | 177.92 | 127.68 | 91.16 |
| | VEGF p = .0173 | | LYVE-1 p = .0001 | |

### Example 4

An optimized assay for identifying subjects with HCC using WGA to detect saccharides on glycosylated proteins is described herein. The assay is an immunological sandwich-based design (FIGS. 4A-4B). Monoclonal antibodies specific to the glycosylated proteins of interest are conjugated to Luminex magnetic beads using a sulfo-NHS bio-conjugation method. There are over 50 different bead sets available at Luminex each with its own unique fluorescent signal. This assay uses three different beads with three unique signals.

A single bead set is chosen for each glycosylated protein of interest and the monoclonal antibodies conjugated accordingly. Beads are then mixed together to form the “multiplex master mix”. The first step in the assay is to incubate a diluted serum sample with the master mix. The protein of interest is captured and all other components in the serum sample are washed away. Care is taken during assay optimization to ensure no antibody-antibody interactions nor non-specific binding of proteins to antibodies takes place in the master mix.

In the second step, the beads, with the protein of interest captured, are incubated with biotinylated WGA or biotinylated polyclonal antibodies (for AFP) to the protein of interest. The highly specific WGA lectin binds N-acetylglucosamine and N-acetylneuraminic acid residues (glycans) on the surface of the captured proteins. Polyclonal antibodies bind several epitopes and allow analysis of the total protein quantity in picograms per milligram in the sample (for AFP only).

The final step is incubation with streptavidin-phycoerythrin (SA-PE). The Luminex MagPix™ is optimized for detection of SA-PE. Signal is obtained for each bead set representing the protein of interest. This signal represents the semi-quantitative amount of saccharides of interest bound to the protein surface. The results are reported in median fluorescent intensity (MFI). Background was low at <10 MFI for all negative control wells. It was determined that blocking of antibody glycans was not necessary for the assay.

AFP is detected with polyclonal antibodies instead of a WGA lectin. The total amount of alpha fetoprotein is assessed in the assay by analysis in a separate microtiter well than the lectin-based detection method.

There is no standard curve for the glycomic assay. Results are reported in raw median fluorescent intensity. This measures the intensity of the signal captured on each magnetic bead for each marker. If results were to be reported in pg/mL, a standard curve would be required. The assay is semi-quantitative.

There is a standard curve for the AFP total assay. It is reported in pg/mL and ranges from 20,000 to the zero standard. Recombinant AFP is used as the standard.

The negative control is the background well for the training set. This consists of PBS only with both primary and secondary antibodies added. Background wells are treated as samples. They are a similar matrix to the samples. Actual patient samples are diluted in PBS in a 1:5 ratio.

The training set used to develop the assay was chosen from the Fried serum bank located within the UNC Liver Center. After patients are consented, samples are collected in serum separator BD tubes, centrifuged and immediately frozen at −80° C.

In practice, HCC typically arises in the background of cirrhosis. It was decided to use patients with cirrhosis as controls in the training set in order to create a more challenging and realistic cohort for finding HCC. Cases had biopsy confirmed HCC. Lesion size was abstracted from medical records for all cases. Lesion size was assessed by MRI.

Selection of cases and controls was not restricted to Hepatitis C positive patients as the test should be applicable across any etiology that places a patient at risk of HCC. Demographics of the training set are presented in Table 8.

TABLE 8
Training Set Demographics

| | | Case | Control |
|---|---|---|---|
| Etiology | | | |
| HCV | | 17 | 22 |
| HBV | | 3 | 1 |
| Alcoholic cirrhosis | | 3 | 6 |
| Co-Infection | | 0 | 3 |
| Other | | 2 | 5 |
| Mean Age | | 57.2 | 58.1 |
| Sex | | | |
| Male | | 19 | 32 |
| Female | | 6 | 5 |
| Race | | | |
| White | | 20 | 28 |
| Black | | 4 | 7 |
| Asian | | 0 | 1 |
| Other | | 1 | 1 |
| HCC Stage | | | |
| Early <3 cm | | 10 | n/a |
| Late >3 cm | | 15 | n/a |
| AFP | | | |
| <20 | ng/mL | 5 | 37 |
| 20-200 | ng/mL | 6 | 0 |
| >200 | ng/mL | 14 | 0 |

| | | |
|---|---|---|
| Days from Dgx* | | |
| <30 | 14 | — |
| <60 | 18 | — |
| <90 | 19 | — |
| <100 | 20 | — |
| <120 | 21 | — |
| <150 | 23 | — |
| <200 | 25 | — |

*includes previous category in cumulative total

WGA lectin was chosen for detection of N-acetylglucosamine and terminal N-acetylneuraminic acid glycan modifications in the final model. The training set consisted of 87 patients with 50 cases and 37 cirrhotic controls. The dependent variable was coded as HCC (cases)=1 and Cirrhosis(controls)=0 and regressed results from the markers in all possible combinations using a logistical binary outcome model with robust standard errors. The algorithm was optimized to get the highest sensitivity noting the trade-off in specificity.

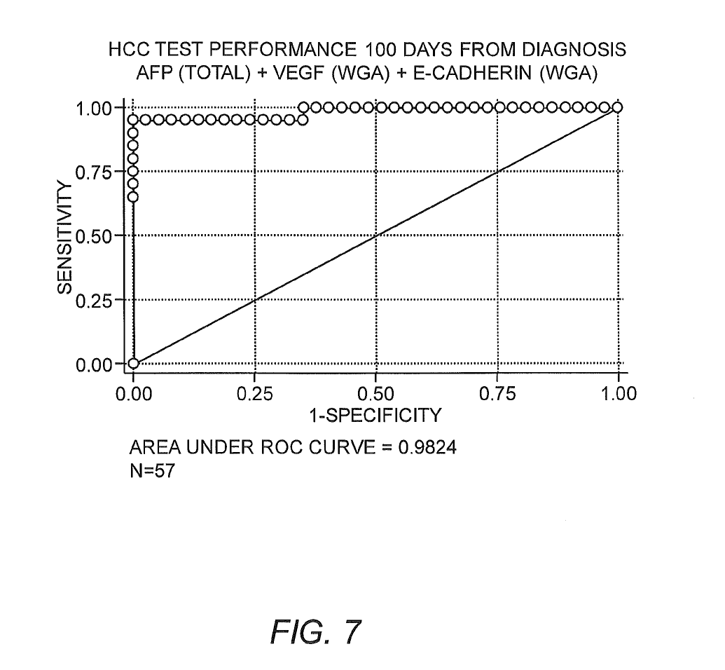

Table 9 and FIG. 7 represent the performance of the selected model that includes total AFP+VEGF(wga)+E-Cadherin(wga) in a logistic regression restricted by days from the date of diagnosis. Controls are included in all restrictions with days from diagnosis coded zero. For example, when restricting the model to days from diagnosis<30, all cases meeting this criterion plus all controls (n=37) are included.

TABLE 9
Final Model Performance Restricted by Days from HCC Diagnosis

| Days from | | | | | | |
|---|---|---|---|---|---|---|
| Dgx* | N* | Sensitivity | Specificity | PPV | NPV | AUROC |
| <30 | 13 | 92.31 | 100 | 100 | 97.37 | 0.9730 |
| <60 | 18 | 94.44 | 100 | 100 | 97.37 | 0.9805 |
| <90 | 19 | 94.74 | 100 | 100 | 97.37 | 0.9815 |
| <100** | 20 | 95.00 | 100 | 100 | 97.37 | 0.9824 |
| <120 | 21 | 90.48 | 100 | 100 | 94.87 | 0.9820 |
| <150 | 23 | 91.30 | 100 | 100 | 94.87 | 0.9835 |
| <200 | 25 | 84.00 | 100 | 100 | 90.24 | 0.9438 |

*cumulative totals and includes only cases meeting the criterion
**best performance achieved within 100 days of HCC diagnosis

To test the robustness of the final model a jackknife test was performed, 16 observations (5 cases/11 controls) were randomly dropped and the model was found to have maintained 93.33% sensitivity with 100% specificity.

The sensitivity of the algorithm at detecting early lesions was tested. Lesion sizes were available for the cases by MRI at initial diagnoses. The model was applied and observations restricted by lesion size as well as days from diagnosis (Table 10).

TABLE 10
Sensitivity of Test to detect early HCC

| Lesion | Days from | | | False | False Negatives |
|---|---|---|---|---|---|
| size | Diagnosis | Sensitivity | Specificity | Positives | (missed HCC) |
| <3 cm | <30 | 80.0 | 100.0 | 0 | 1 |
| (n = 42) | | | | | |
| <3 cm | <60 | 85.71 | 100.0 | 0 | 1 |
| (n = 44) | | | | | |
| <3 cm | <90 | 85.71 | 100.0 | 0 | 1 |
| (n = 44) | | | | | |
| <3 cm | <100 | 85.71 | 100.0 | 0 | 1 |
| (n = 44) | | | | | |
| <3 cm | <120 | 75.00 | 100.0 | 0 | 2 |
| (n = 45) | | | | | |
| <3 cm | <150 | 77.78 | 100.0 | 0 | 2 |
| (n = 46) | | | | | |
| <3 cm | <200 | 77.78 | 100.0 | 0 | 2 |
| (n = 46) | | | | | |

Note:
N includes controls + cases jointly meeting the lesion size and days from diagnosis criteria

To cross-validate the model, a k-fold function was used in Stata. This command performs a k-fold cross-validation on a specified model in order to evaluate a model's ability to fit out-of-sample data. This procedure splits the data randomly into k partitions, then for each partition it fits the specified model using the other k-1 groups and uses the parameters to make predictions in the unused group. The analysis yielded 93.75% sensitivity and 100% specificity estimations for out-of-sample data with AUROC=0.9771. 97.83% of the training set was classified correctly at 100 days from HCC diagnosis. This indicates the model is robust and has the ability to fit out-of-sample data very well.

### Example 5

Methods similar to the one described in Example 4 were applied to different combinations of glycosylated proteins in combination with different plant lectins. The results are shown in Tables 11-13. The data indicate that the method is effective with multiple plant lectins, including WGA (which binds to N-acetylneuraminic acid and N-acetylglucosamine), SBA (which binds to N-acetylgalactosamine), and DBA (which binds to N-acetylgalactosamine). The data further indicate that many different combinations of glycosylated proteins, with as few as 1 and as many as 5, are effective, when combined with total AFP, to produce results with both high sensitivity and high specificity.

### Example 6

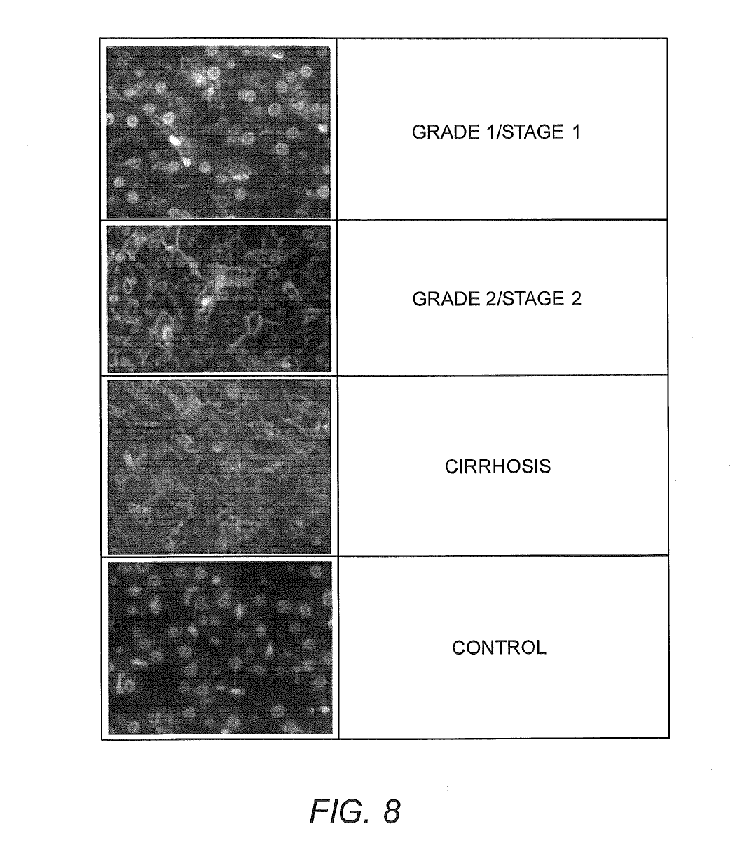

A histological analysis of glycosylation levels in tissue samples from liver was carried out. Tissue specimens were obtained from biopsies and explanted liver segments. All samples were received as 4 μm formalin fixed paraffin-embedded slices mounted on glass slides. Samples were from three different patients. All had chronic hepatitis C virus with varying degrees of liver fibrosis and one had cirrhosis. Slides were stained with wheat germ agglutinin-rhodamine and DAPI and viewed with a fluorescence microscope. Various grades of fibrosis up to an including cirrhosis were analyzed (FIG. 8). There were noticeable increases in the binding of WGA to either N-acetylneuraminic acid or N-acetylglucosamine in the tissue with increasing stages of disease, indicating that this method can be used to stage liver disease.

TABLE 11
Glycosylated protein combinations with WGA lectin and AFP

| WGA Lectin Results netMFI | | | | | Performance Parameters | | | | | | | |
|---|---|---|---|---|---|---|---|---|---|---|---|---|
| | Glypican- | Galectin- | E- | LYVE- | | | | | | % correctly | False | False |
| VEGF | 3 | 3 | Cadherin | 1 | AUROC | Sensitivity | Specificity | NPV | PPV | classified | Negative | Positive |
| X | X | X | X | X | 0.9919 | 95 | 100 | 97 | 100 | 98 | 1 | 0 |
| X | X | X | X | | 0.9797 | 95 | 100 | 97 | 100 | 98 | 1 | 0 |
| X | X | X | | | 0.9635 | 95 | 100 | 97 | 100 | 98 | 1 | 0 |
| X | X | | | | 0.9635 | 95 | 100 | 97 | 100 | 98 | 1 | 0 |
| X | | | | | 0.9622 | 95 | 100 | 97 | 100 | 98 | 1 | 0 |
| | | | | X | 0.9527 | 90 | 100 | 94 | 100 | 96 | 2 | 0 |
| | | | X | X | 0.9892 | 95 | 100 | 97 | 100 | 98 | 1 | 0 |
| | | X | X | X | 0.9892 | 95 | 100 | 97 | 100 | 98 | 1 | 0 |
| | X | X | X | X | 0.9892 | 95 | 100 | 97 | 100 | 98 | 1 | 0 |
| X | | | | X | 0.9824 | 95 | 100 | 97 | 100 | 98 | 1 | 0 |
| X | | | X | | 0.9824 | 95 | 100 | 97 | 100 | 98 | 1 | 0 |
| X | | X | | | 0.9635 | 95 | 100 | 97 | 100 | 98 | 1 | 0 |
| | X | | | X | 0.9757 | 90 | 100 | 94 | 100 | 96 | 2 | 0 |
| | X | | X | | 0.9730 | 95 | 100 | 97 | 100 | 98 | 1 | 0 |
| | X | X | | | 0.9622 | 95 | 100 | 97 | 100 | 98 | 1 | 0 |
| | | X | | X | 0.9851 | 90 | 100 | 94 | 100 | 96 | 2 | 0 |
| | | X | X | | 0.9784 | 95 | 100 | 97 | 100 | 98 | 1 | 0 |
| | | | X | X | 0.9892 | 95 | 100 | 97 | 100 | 98 | 1 | 0 |
| X | | | | | 0.9622 | 95 | 100 | 97 | 100 | 98 | 1 | 0 |
| | X | | | | 0.9662 | 90 | 100 | 94 | 100 | 96 | 2 | 0 |
| | | X | | | 0.9595 | 90 | 100 | 94 | 100 | 96 | 2 | 0 |
| | | | X | | 0.9689 | 95 | 100 | 97 | 100 | 98 | 1 | 0 |
| | | | | X | 0.9527 | 90 | 100 | 94 | 100 | 96 | 2 | 0 |

TABLE 12
Glycosylated protein combinations with SBA lectin and AFP

| SBA Lectin | | Performance Parameters | | | | | | | |
|---|---|---|---|---|---|---|---|---|---|
| Results net MFI | | | | | | | % correctly | False | False |
| VEGF | LYVE-1 | AUROC | Sensitivity | Specificity | NPV | PPV | classified | Negative | Positive |
| X | X | 0.9722 | 89 | 100 | 94 | 100 | 96 | 2 | 0 |
| X | | 0.9488 | 89 | 100 | 94 | 100 | 96 | 2 | 0 |
| | X | 0.9708 | 89 | 100 | 94 | 100 | 96 | 2 | 0 |

TABLE 13
Glycosylated protein combinations with DBA lectin and AFP

| DBA Lectin | | Performance Parameters | | | | | | | |
|---|---|---|---|---|---|---|---|---|---|
| Results net MFI | | | | | | | % correctly | False | False |
| VEGF | LYVE-1 | AUROC | Sensitivity | Specificity | NPV | PPV | classified | Negative | Positive |
| X | X | 0.9929 | 89 | 100 | 95 | 100 | 96 | 2 | 0 |
| X | | 0.9687 | 89 | 100 | 94 | 100 | 96 | 2 | 0 |
| | X | 0.9929 | 91 | 97 | 94 | 95 | 95 | 2 | 1 |

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A method for detecting the presence of a liver disease in a subject, comprising:
(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of alpha fetoprotein (AFP) in the biological sample; and
(c) determining that a liver disease is present based on the level of saccharide and amount of AFP.

2. The method of claim 1, wherein the liver disease is hepatocellular carcinoma, cirrhosis, organ transplant rejection, veno-occlusive disease, or sinusoidal obstruction syndrome.

3-7. (canceled)

8. A method for staging a liver disease in a subject, comprising:
(a) measuring the level of a saccharide selected from N-acetylneuraminic acid, N-acetylglucosamine, N-acetylgalactosamine, or any combination thereof on one or more glycosylated proteins in a biological sample from a subject;
(b) measuring the amount of AFP in the biological sample; and
(c) staging a liver disease in the subject based on the level of saccharide and amount of AFP.

9. The method of claim 8, wherein the liver disease is hepatocellular carcinoma, cirrhosis, or fibrosis.

10-13. (canceled)

14. The method of claim 1, wherein the level of a saccharide is measured on two or more glycosylated proteins.

15. The method of claim 1, wherein the level of a saccharide is measured on three or more glycosylated proteins.

16. The method of claim 1, wherein the glycosylated proteins are one or more proteins selected from the list in Table 1 or any combination thereof.

17. The method of claim 1, wherein the glycosylated proteins are one or more proteins selected from vascular endothelial growth factor (VEGF), lymphatic vessel endothelial hyaluronan receptor (LYVE-1), E-cadherin, alpha 1 acid glycoprotein, glypican-3, galectin-3, or any combination thereof.

18-19. (canceled)

20. The method of claim 1, wherein the level of a saccharide selected from N-acetylneuraminic acid, N-acetylglucosamine, and/or N-acetylgalactosamine is measured using a ligand that specifically binds to one or more of N-acetylneuraminic acid, N-acetylglucosamine, and/or N-acetylgalactosamine.

21-23. (canceled)

24. The method claim 1, wherein the biological sample is serum, plasma, urine, or tissue.

25. The method of claim 1, wherein the determining step is carried out using an empirically-based regression algorithm.

26. The method of claim 25, wherein the determining step further comprises assigning a weighted coefficient to the levels of a saccharide selected from N-acetylneuraminic acid, N-acetylglucosamine, and/or N-acetylgalactosamine.

27. The method of claim 1, wherein the determining step comprises comparing the level of a saccharide selected from N-acetylneuraminic acid, N-acetylglucosamine, and/or N-acetylgalactosamine in the biological sample to the level of N-acetylneuraminic acid, N-acetylglucosamine, and/or N-acetylgalactosamine in a control.

28. The method of claim 1, wherein the measuring steps are carried out using an immunoassay.

29. The method of claim 1, wherein the measuring steps are carried out using a liquid-based assay.

30-32. (canceled)

33. The method of claim 1, providing a sensitivity of at least 80%.

34. The method of claim 1, providing a specificity of at least 80%.

35-39. (canceled)

40. The method of claim 1, further comprising the step of treating the liver disease if present in the subject.

41. The method of claim 1, further comprising the step of administering to the subject an additional test for the liver disease.

42-49. (canceled)

50. A kit for carrying out a method according to claim 1, the kit comprising:
(a) one or more reagents for measuring the level of a saccharide selected from N-acetylneuraminic acid, N-acetylglucosamine, N-acetylgalactosamine, or any combination thereof on one or more glycosylated proteins in a biological sample from a subject; and
(b) one or more reagents for measuring the amount of AFP in a biological sample from a subject.

51-66. (canceled)
